SEQUENCE LISTING (12 sequences)

SEQ ID NO: 1 - RNA, 774 nt, Homo sapiens
uuauguaucauacacauacgauuuaggugacacuauagaauacaagcuuaugcaugcggccgcaucuagagggcccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaaugugacaagccgaggcggugagccgggcaggaggaaggagccucccucaggguuucgggaaccagauccacuaguucuagaugcaugcucgagcggccgccagugugauggauaucugcagaauuccagcacacuggccguuacuaguggauccgagcucccaaaaaaaaaaaaaaaaaaaaaaaaaaaaaaccgaauuaauucguaaucauggucauagcuguuuccugugugaaauuguuauccgcuca

SEQ ID NO: 2 - RNA, 1873 nt, Homo sapiens
ucgcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaauuaa

SEQ ID NO: 3 - DNA, 20 bp, Artificial Sequence
  synthetically generated oligonucleotide
ctagactgtgtgttctggag

SEQ ID NO: 4 - DNA, 20 bp, Artificial Sequence
  synthetically generated oligonucleotide
acctctttcctctttctgct

SEQ ID NO: 5 - DNA, 20 bp, Artificial Sequence
  synthetically generated oligonucleotide
ctctctcttcctcgacttct

SEQ ID NO: 6 - DNA, 20 bp, Artificial Sequence
  synthetically generated oligonucleotide
accccgtctctctcttcctc

SEQ ID NO: 7 - DNA, 20 bp, Artificial Sequence
  synthetically generated oligonucleotide
ctcctcttccttctcttctt

SEQ ID NO: 8 - DNA, 21 bp, Artificial Sequence
  synthetically generated oligonucleotide
gttctgtatcagtctttcctg

SEQ ID NO: 9 - DNA, 24 bp, Artificial Sequence
  synthetically generated oligonucleotide
cttcatttcaggtttctggattaa

SEQ ID NO: 10 - DNA, 20 bp, Artificial Sequence
  synthetically generated oligonucleotide
tctttctttggtctgcattc

SEQ ID NO: 11 - RNA, 1150 nt, Homo sapiens
gaauacaagcuuaugcaugcggccgcaucuagagggcccggauccaaauggaagacgccaaaaacauaaagaaaggcccggcgccauucuauccucuagaggauggaaccgcuggagagcaacugcauaaggcuaugaagagauacgcccugguuccuggaacaauugcuuuuacagaugcacauaucgaggugaacaucacguacgcggaauacuucgaaauguccguucgguuggcagaagcuaugaaacgauaugggcugaauacaaaucacagaaucgucguaugcagugaaaacucucuucaauucuuuaugccgguguugggcgcguuauuuaucggaguugcaguugcgcccgcgaacgacauuuauaaugaacgugaauugcucaacaguaugaacauuucgcagccuaccguaguguuuguuuccaaaaagggguugcaaaaaauuuugaacgugcaaaaaaaauuaccaauaauccagaaaauuauuaucauggauucuaaaacggauuaccagggauuucagucgauguacacguucgucacaucucaucuaccucccgguuuuaaugaauacgauuuuguaccagaguccuuugaucgugacaaaacaauugcacugauaaugaauuccucuggaucuacuggguuaccuaaggguguggcccuuccgcauagaacugccugcgucagauucucgcaugccagagauccuauuuuuggcaaucaaaucauuccggauacugcgauuuuaaguguuguuccauuccaucacgguuuuggaauguuuacuacacucggauauuugauauguggauuucgagucgucuuaauguauagauuugaagaagagcuguuuuuacgaucccuucaggauuacaaaauucaaagugcguugcuaguaccaacccuauuuucauucuucgccaaaagcacucugauugacaaauacgauuuaucuaauuuacacgaaauugcuucugggggcgcaccucuuucgaaagaagucggggaagcgguugcaaaacgcuuccaucuuccagggauacgacaaggauaugggcucacugagacuacaucagcuauucugauuacacccgagggggaugauaaaccgggcgcggucgguaaaguuguuccauu

SEQ ID NO: 12 - DNA, 20 bp, Artificial Sequence
  synthetically generated oligonucleotide
cattatcagtgcaattgttt

(19) United States
(12) Patent Application Publication
(10) Pub. No.: US 2005/0261485 A1
(43) Pub. Date: Nov. 24, 2005

(54) Method of producing antisense oligonucleotide

(75) Inventor: Kiyoshi Uchida, Ibaraki (JP)

Correspondence Address: FISH & RICHARDSON, PC, 12390 EL CAMINO REAL, SAN DIEGO, CA, 92130-2081, US

(73) Assignee: Toagosei Co., Ltd., a Japan corporation

(21) Appl. No.: 10/611,823

(22) Filed: Jun. 30, 2003

Related U.S. Application Data
(62) Division of application No. 08/859,415, filed on May 20, 1997, now abandoned.

(30) Foreign Application Priority Data
May 23, 1996 (JP) 128192/1996

(51) Int. Cl. C12Q 1/68; G06F 19/00; G01N 33/48; G01N 33/50; C07H 21/04

(52) U.S. Cl. 536/23.1; 435/6; 702/20

(57) ABSTRACT

The object of the present invention is to provide a method of producing antisense oligonucleotide, in which the possibility of forming a substantially complementary double-stranded chain between each region of a nucleotide sequence in mRNA and a region other than said region is expressed as a numerical value, and oligonucleotide substantially complementary to a region with a smaller numerical value is prepared as antisense oligonucleotide. The resulting antisense oligonucleotide can be used effectively in the antisense oligonucleotide method.

### FIELD OF THE INVENTION

The present invention relates to a method of producing antisense oligonucleotide.

### BACKGROUND OF THE INVENTION

The antisense oligonucleotide method is a method of inhibiting the expression of a protein by hybridizing, to a target gene, oligonucleotide (antisense oligonucleotide) having a sequence complementary or substantially complementary to the target gene, and this method is used in e.g. the preparation of medical drugs or genetic recombinant plants.

In the antisense oligonucleotide method, it is first necessary to prepare oligonucleotide with a nucleotide sequence having an inhibitory effect (antisense oligonucleotide effect or antisense effect) on expression of a gene by hybridization to the gene. Preparation of oligonucleotide with such a nucleotide sequence includes a method of synthesizing oligonucleotide complementary to experimentally found antisense-effective region and a method of synthesizing oligonucleotide complementary to predicted antisense-effective region without experiment.

In the former method, however, it is necessary to prepare oligonucleotides whose sequences are complementary to a target gene and to experimentally determine whether they show antisense effect on the target gene, which results in a long period of time, higher costs and complicated procedures.

In the latter method, an initiation site for translation, its upstream non-coding region, or other region are selected empirically as a target site and then antisense oligonucleotides to the target site are prepared. Concerning the latter method, various methods are proposed (K. R. Blake et al., Biochemistry, 24, 6132-6138 (1985); E. Uhlmann, A. Peyman, Chemical Reviews, 90, 543-584 (1990)), but their reliabilities are low and the antisense oligonucleotides selected in these methods do not always effectively inhibit the expression of the target protein (e.g. R. D. Ricker, A. Kaji, FEBS Letters, 309, 363-370 (1992). There are also proposals for predicting a target site of antisense oligonucleotide based on calculation of energy for formation of secondary structure of mRNA or its precursor, or energy for formation of a hybrid between MRNA or its precursor and antisense oligonucleotide, or the difference between these two energies. However, antisense oligonucleotides selected in these methods are low in reliability and are not always effective (R. A. Stull et al., oligonucleotide Research, 20, 3501-3508 (1992)).

In the antisense oligonucleotide method, therefore, there is demand for highly reliable prediction of an effective antisense oligonucleotide, that is, antisense oligonucleotide having a nucleotide sequence to effectively inhibit expression of mRNA (or its precursor) coding for a target protein, in order to facilitate the preparation of antisense oligonucleotide.

### SUMMARY OF THE INVENTION

The object of the present invention is to provide a method of producing antisense oligonucleotide in which the antisense oligonucleotide can be obtained efficiently without conducting any experiment.

As a result of eager research, the present inventor found that antisense oligonucleotide can be prepared as the following. A possibility of forming a substantially complementary duplex between a specific region in mRNA and every other region in the same mRNA is calculated and oligonucleotide having a substantially complementary sequence to the specific region with a low value for the sum of the duplex-forming possibility is prepared.

That is, the present invention is a method of producing antisense oligonucleotide, in which the possibility of forming a substantially complementary double-stranded chain between each region of a nucleotide sequence in MRNA and a region other than said region is expressed as a numerical value, and oligonucleotide substantially complementary to a region with a smaller numerical value is prepared as antisense oligonucleotide.

The above possibility is numerically expressed on the basis of distance between two nucleotide sequence regions forming a substantially complementary double-stranded chain, and more specifically the possibility is expressed as a lower value as the distance increases, and it is expressed as the largest numerical value when there are 3 to 10 bases, preferably 4 to 6 bases between the two nucleotide sequence regions. Further, the possibility is expressed on the basis of the bond energy for forming a double-stranded chain, and more specifically it is expressed as a larger numerical value when the bond energy is higher. The bond energy herein used can be obtained using the nearest neighbor model.

In particular, the above possibility is expressed most preferably as a numerical value based on the distance between the two nucleotide sequence regions and the bond energy for forming a substantially complementary double-stranded chain.

### BRIEF DESCRIPTION OF THE DRAWINGS

FIG. 1 shows an outline of a method of calculating the possibility of forming a double-stranded chain of mRNA.

FIGS. 2 (A) to (C) show an outline of a method of calculating the possibility of forming a double-stranded chain of mRNA.

FIGS. 3 (A) and (B) show an outline of a method of calculating the possibility of forming a double-stranded chain of mRNA.

FIG. 4 shows a flow chart for a method of calculating the possibility of forming a double-stranded chain of mRNA.

FIG. 5 shows results for the calculated possibility of VEGF mRNA to form a substantially complementary double-stranded chain and results for experimentally-determined expression of VEGF.

FIG. 6 shows the expression of VEGF in the presence of antisense oligonucleotide in a cell-free transcription and translation system.

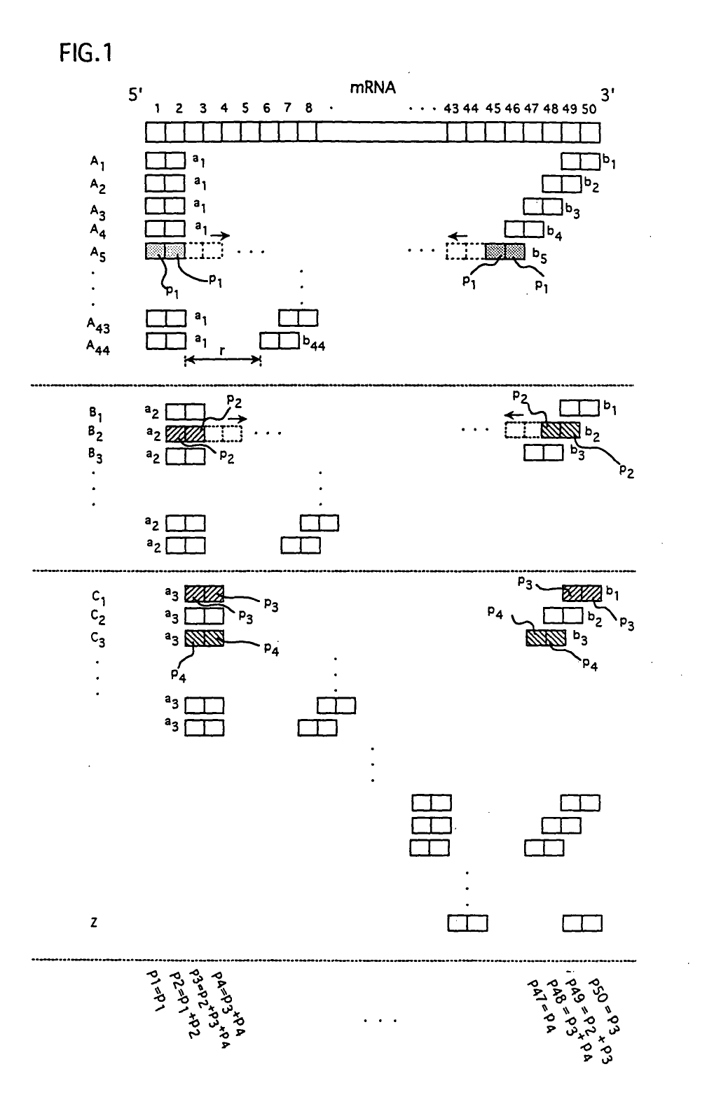

In FIGS. 2 and 3, 1 is a loop, 2 is a loop, 3 is a stem, 4 is a stem, 5 is a loop, and 6 is a loop.

### DETAILED DESCRIPTION OF THE INVENTION

Hereinafter, the present invention is described in detail.

mRNA can form a double-stranded chain if a certain specific region is substantially complementary to another region in the same MRNA. This property is utilized in the present invention to prepare antisense oligonucleotide as the following. A possibility of forming a substantially complementary chain between a specific region in mRNA and every other region in the same mRNA is evaluated numerically and then an antisense oligonucleotide substantially complementary to a region assigned a summed low numerical value as this possibility is prepared, assuming that a region having a summed high numerical value is readily forming a substantially complementary chain while a region having a substantially complementary sequence to a summed low numerical value is hardly forming a substantially complementary chain, i.e. the region is remaining in a single-stranded chain.

In the present invention, the possibility of forming a substantially complementary chain (also referred to hereinafter as “the ability to form a substantially complementary chain”) is numerically expressed. The term “complementary” refers to the state in which a specific region in mRNA and another specific region in the same RNA hybridize to each other (i.e. forming a base pair), and for example, it refers to the relationship between adenine (A) and uracil (U) or the relationship between cytosine (C) and guanine (G). In the present invention, the term “complementarity” means that a specific region and another region in the same mRNA hybridize to each other with antiparallel direction, that is, a certain region in the 5′→3′ direction of the sequence hybridizes to another region in the 3′→5′ direction of the sequence.

Further, with the term “substantially” given, the base pair in the present invention is not limited to the base pair of G and C or the base pair of A and U, and it includes any other base pair if these bases hybridize to each other. Therefore, the base pair can include not only the base pair of G and C or A and U but also the base pair of G and U. Furthermore, if the substantially complementary chain is sufficiently long (e.g. 10 bases or more), a few mismatch base pairs may occur.

Hereinafter, the specific means of numerically expressing the ability to form a substantially complementary double-stranded chain is described.

First, whether a region beginning at a base at a specific site and consisting of continuous 1 or more bases, preferably 2 to 4 or more bases, in a specific direction (a predetermined direction of 5′→3′ or 3′→5′) in the mRNA (or its precursor), is substantially complementary to a region apart by 4 bases or more (preferably 5 bases or more) from said region is determined by examining the whole nucleotide sequence of the RNA. If they are substantially complementary, it is determined whether their respective next bases are substantially complementary to each other, and if they are still substantially complementary, their further next bases are examined for substantial complementarity. In this manner, a possible maximum substantially complementary region is identified. This region containing the specific site is a specific region in the mRNA. Then, the possibility that this specific region and its corresponding substantially complementary region form a substantially complementary double-stranded chain is numerically expressed. The computer program for this is set such that insofar as they are apart from each other by a predetermined distance, a larger numerical value is given for a shorter distance between the substantially complementary regions and a smaller numerical value is given for a longer distance. Also, the calculation program is set such that a larger numerical value is given when the energy for forming a double-stranded chain is larger and a smaller numerical value is given when the energy is smaller. The value thus numerically expressed is assigned to each base in said specific region to form said double-stranded chain (and also to each base in said substantially complementary region to form a double-stranded chain).

Then, a next other region substantially complementary to said region beginning at the specific site is identified in the same manner as described above, and the ability of the identified complementary region and said specific region to form a substantially complementary double-stranded chain is numerically expressed in the same manner. Then, this numerical value is added to the previously determined numerical value assigned to each base. In this manner, a region beginning at the specific site in mRNA is examined for its substantial complementarity to every other region in the same mRNA, and if they are complementary, their ability to form a substantially complementary double-stranded chain is numerically expressed in the same manner as above. This numerical value is assigned to the corresponding base (if there is a previous value assigned to the base, it is assigned to the base by adding it to the previous value). If the computer program is set such that this numerical value is assigned to both the specific region and its substantially complementary region, the value is also assigned to the latter region in the same manner.

Every possible region in the mRNA is examined in the same manner, and the ability of each region to form a substantially complementary double-stranded chain is determined as the sum of the numerical values thus obtained for each base. To determine the sum of the numerical values for each base, the numerical value obtained between certain bases and their corresponding complementary bases within a substantially complementary region shall not be added twice or more times. For example, let us suppose a region of 2 bases or more beginning at the 10-position towards the 3′-terminal (where the 5′-terminal is given the 1-position) is examined as a specific region for its substantial complementarity to 2 bases or more beginning at the 30-position towards the 5′-terminal. If it is assumed that 4 bases beginning at the 9-position towards the 3′-terminal are found to be substantially complementary to 4 bases beginning at the 31-position towards the 5′-terminal, then a numerical value obtained between 3 bases beginning at the 10-position towards the 3′-terminal and 3 bases beginning at the 30-position towards the 5′-terminal or a numerical value obtained between 2 bases beginning at the 11-position towards the 3′-terminal and 2 bases beginning at the 29-position towards the 5′-terminal are not counted as the ability to form a substantially complementary double-stranded chain.

If a certain specific region is substantially complementary to another region, the possibility of forming a double-stranded chain by these 2 regions is expressed with a significant value, while if a certain specific region is not substantially complementary to another region, the possibility of forming a double-stranded chain by these 2 regions is expressed as zero (0). For this purpose, we can use a computer programme to calculate the possibility.

The type of mRNA to which the antisense oligonucleotide is prepared according to the present method, is not particularly limited. Examples of such mRNA are mRNA for vascular endothelial growth factor derived from animals such as humans or rats, mRNA for heme oxygenase I type or II type derived from animals such as humans or rats, and mRNA for luciferase derived from firefly.

The method of numerically expressing the possibility of forming a double-stranded chain by a part of the sequence of mRNA and a different part in the mRNA is outlined in FIG. 1.

To examine a specific region in mRNA for complementarity, the length of its fragment is preferably 2 to 4 base pairs or more. FIG. 1 shows the case where the whole length of mRNA is 50 bases and the length of a specific region is 2 bases or more. In FIG. 1, it is determined whether 2 bases (bases at the 1- and 2-positions, referred to as a₁) at the 5′-terminal in MRNA and 2 bases (bases at the 50- and 49-positions, referred to as b₁) at the 3′-terminal in the same mRNA are substantially complementary (A₁). If a₁ and b₁ are not substantially complementary, then it is determined whether a₁ is complementary to 2 bases (bases at the 49- and 48-positions, referred to as b₂) shifted by 1 base to the 5′-terminal from b₁ (FIG. 1, A₂). This procedure is repeatedly carried out to determine whether there is a sequence complementary to a₁ in the whole region of mRNA except for region required for minimum distance (3 bases between the two chains) (FIG. 1, A₁ to A₄₄). If a₁ is found to be complementary to 2 bases (the 46- and 45-positions, referred to as b₅) apart by 4 bases from the 3′-terminal (FIG. 1, A₅), then their next bases, i.e. a base at the 3-position and a base at the 44-position, are examined for substantial complementarity. If said 2 bases are found to be substantially complementary to each other, their next bases, i.e. a base at the 4-position and a base at the 43-position are further examined for substantial complementarity (see “→” and “←” in A₅, FIG. 1). The same procedure is repeatedly carried out insofar as substantially complementary bases continue (provided that a minimum number of bases required for forming a loop between the substantially complementary bases are secured), and the ability of the complementary nucleotide sequences to form a double-stranded chain is numerically expressed. This numerical value is given to each base in a₁ forming a complementary nucleotide sequence beginning at the 1-position (and to each base in b₅ which forms a substantially complementary double-stranded chain with a₁). Bond energy can be further used as a numerical value for reflecting the ability of said complementary base sequences to form a double-stranded chain, as described below:

It can be assumed here that a higher ability of forming a substantially complementary double-stranded chain between a specific site and its corresponding substantially complementary site leads to formation of a more stable substantially complementary chain.

The ability to form a substantially complementary double-stranded chain depends on experimentally-determined bond energy (Gibbs free energy) for formation of a substantially complementary double-stranded chain or calculated bond energy based on the nearest neighbor model. Thus, the ability to form a substantially complementary double-stranded chain is numerically expressed by using such values.

For example, the ability to form a substantially complementary double-stranded chain can be numerically expressed by using the “nearest neighbor model” (Naoki Sugimoto, “Seibutsubutsuri” (Biophysics), Vol. 33, pp. 1-7, (1993)) in the manner as described below. The “nearest neighbor model” is a model made on the ground that the formation of a base pair is most influenced by its next and previously formed base-pair. Bond energy ΔG generated between a dimer (i.e. consisting of a certain base and its next base) and its substantially complementary sequence is expressed in Table 1 below.

TABLE 1

| Sequence | |
|---|---|
| (sense chain/ | |
| antisense chain) | ΔG |
| 5′→3′ 3′ [symbol] 5′ | (kcal/mol) |
| AA/UU | −0.7 |
| UU/AA | −0.7 |
| AU/UA | −0.8 |
| CG/GC | −1.9 |
| CU/GA | −1.6 |
| AG/UC | −1.6 |
| GA/CU | −2.1 |
| UC/AG | −2.1 |
| GC/CG | −3.2 |
| GG/CC | −2.8 |
| CC/GG | −2.8 |
| GU/CA | −2.0 |
| AC/UG | −2.0 |
| UA/AU | −0.9 |
| UG/AC | −1.8 |
| CA/GU | −1.8 |
| AG/UU | −0.4 |
| UU/GA | −0.4 |
| CG/GU | −1.3 |
| UG/GC | −1.3 |
| GG/CU | −1.2 |
| UC/GG | −1.2 |
| UG/AU | −0.5 |
| UA/GU | −0.5 |
| GG/UU | −0.4 |
| UG/GU | −0.5 |
| AU/UG | −0.5 |
| GU/UA | −0.5 |
| CU/GG | −1.3 |
| GG/UC | −1.3 |
| GU/CG | −1.7 |
| GC/UG | −1.7 |
| UU/AG | −0.4 |
| GA/UU | −0.4 |
| GU/UG | −0.4 |
| UU/GG | −0.4 |

The ability to form a substantially complementary double-stranded chain can be calculated using both the bond energy of corresponding base pairs in Table 1 and the distance between 2 strands in the double-stranded chain in the following equation: The ability to form a substantially complementary double-stranded chain=((L+1)/r)^{F}·exp (|ΔG|/RT) (I) where |ΔG| is absolute value of bond energy, R is gas constant, T is absolute temperature, L is minimum number of bases in a loop, r is distance between one of double strands and another strand (number of bases between 2 chains (regions)+1), and F is evaluation index for distance r.

L is in the range of 3 to 10, preferably 4 to 6. r is an integer of 1 or more. F is zero or a positive number which can be assigned e.g. 6, ⅓, 0.1, etc.

For example, if a specific site is located at the 1-position and there is the sequence “GUAU” at the 43- to 46-positions substantially complementary to the nucleotide sequence “AUGC” at the 1- to 4-positions, then the bond energy ΔG for their formation of a double-stranded chain can be calculated on the basis of Table 1, as follows:

$$\begin{matrix}Δ G = Δ ( AU / UA ) + Δ ( UG / AU ) + Δ ( GC / UG ) \\ = ( - 0.8 ) + ( - 0.5 ) + ( - 1.7 ) \\ = - 3.0\end{matrix}$$

Also, r=43−4=39. If L=4, F=6, and the temperature is 37° C. (T=310.15K), then the ability to form a substantially complementary double-stranded chain in this case can be numerically expressed as follows: The ability to form a substantially complementary double-stranded chain (1-4 and 46-43)

$$\begin{matrix} = {( 5 / 39 )}^{6} \cdot exp ( 3.0 kcal \text{/} RT ) \\ = {( 5 / 39 )}^{6} \cdot exp ( 3000 / 1.9872 \times 310.15 ) \\ = 5.77 \times {10}^{- 4}\end{matrix}$$

The resulting numerical value (5.77×10⁻⁴) is given to the respective bases at the 1- to 4-positions and the 43- to 46-positions.

It is thereafter a region complementary (or substantially complementary) to a nucleotide sequence of at least 2 bases beginning at the 1-position as the specific site is searched, and if present, its ability to form a (substantially) complementary double-stranded chain is calculated. For example, if the sequence at the 39- to 40-positions is “AU” and the sequence at the 15- to 17-positions is “CAU” in the above example, the bases “AU” at the 1- to 2-positions are complementary to the bases “AU” at the 39- to 40-positions and the bases “AUG” at the 1- to 3-positions are complementary to the bases “CAU” at the 15- to 17-positions. Hence, the ability to form a substantially complementary double-stranded chain is numerically expressed for each case, and the numerical value for the former (double-stranded chain formed with bases at the 39- to 40-positions) is added to bases at the 1- to 2-positions (and bases at the 39- to 40-positions) and the numerical value for the latter (double-stranded chain formed with bases at the 15-to 17-positions) is added to bases at the 1- to 3-positions (and bases at the 15- to 17-positions).

This operation is continued until the distance between the 3′-side base in the specific region (a₁) and the 5′-side base in other regions (b₁, b₂, . . . ) reaches a predetermined number of bases (FIG. 1, A₄₄). The minimum value of “r” may be arbitrarily set e.g. at 4 to 11, preferably 5 to 7.

In the above example, minimum “r” was set as 4. Thus, the complementarity to the sequence of a₁ is examined for sequences of b₁, b₂, . . . to the sequence at the 7- to 6-positions (FIG. 1, A₄₄).

In this way, a specific region beginning at a specific base (in this case, the 1-position) is examined for substantial complementarity to continuous 2 or more bases throughout the whole region of mRNA (excluding a region located with a distance of less than “r” between a specific region beginning at a specific base (in this case, the 1-position) and its corresponding complementary region), and if there is a substantially complementary region, the ability to form a substantially complementary chain is numerically expressed and this numerical value is assigned to each base in the specific region (and each base in its corresponding substantially complementary chain) or added to the value of a previous value if any.

Then, whether a sequence (bases at the 2- and 3-positions, referred to as a₂) apart by 1 base from the 5′-terminal of the mRNA is substantially complementary to 2 bases (bases at the 50- to 49-positions, referred to as b₁) from the 3′-terminal of the same mRNA is examined (FIG. 1, B₁). In the same manner as above, the sequence of a₂ is examined for substantial complementarity to the sequence of b₁, b₂, b₃ . . . which are apart by 1 base from one another. If there is a substantially complementary region, substantial complementarity of their next bases is then determined as described above. For example, as shown in B₂, whether a base next to a₂ (base at the 4-position from the 5′-terminal) is substantially complementary to a base at the 47-position (base at the 4-position from the 3′-terminal) in b₂ is examined. If they are substantially complementary, whether their next bases (i.e. base 5 in a₂ and base 46 in b₂) are substantially complementary and so forth (see arrows in B₂ in FIG.1).

This procedure is repeatedly carried out, and the possibility of forming a substantially complementary chain is numerically expressed for each substantially complementary region (i.e. A₅, B₂, C₁, and C₃ in FIG. 1), and this numerical value is assigned to each base in the specific region (and each base in its corresponding chain) forming a substantially complementary double-stranded chain to finish the procedure (FIG. 1, Z).

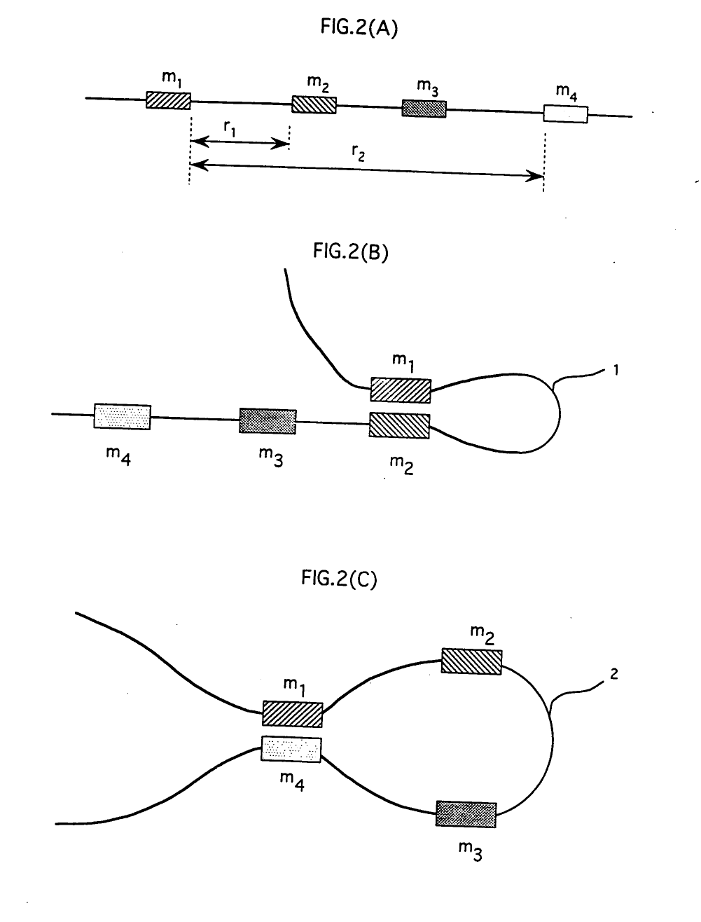

The computer program for numerically expressing the possibility of forming a substantially complementary double-stranded chain is set such that the numerically expressed possibility of forming a substantially complementary chain is assigned to one strand (region) and/or another strand (region) forming a double-stranded chain. In the above example (FIG. 1), the numerically expressed possibility of forming a substantially complementary chain is assigned to both of the strands forming a double-stranded chain. If the numerically expressed possibility of forming a substantially complementary chain is assigned to either (region) of the strands in FIG. 1, the value may be assigned to the regions a₁, a₂, a₃, . . . , or alternatively to the regions b₁, b₂, b₃, . . . . In other words, if the value is assigned to only the regions a₁, a₂, a₃, . . . , the value is not assigned to the regions b₁, b₂, b₃, . . . . If the numerically expressed possibility of forming a substantially complementary chain is assigned to only one (region) of the strands, whether a nucleotide sequence at the 2- to 1-positions is complementary to a nucleotide sequence at the 49- to 50-positions as the specific region is determined, and if they are substantially complementary, the possibility of forming a double-stranded chain is numerically expressed and then assigned to bases at the 49- and 50 -position by adding the value to a previous value if any. To evaluate the possibility of forming a substantially complementary chain, the computer program is set such that the possibility of forming a substantially complementary chain is expressed as a smaller value when the “r” value is larger. For example, if in FIG. 2(A), m₁ is substantially complementary to m₂ and m₄, the ability of m₁ and m₂ to form a substantially complementary chain is calculated to be higher or not lower than that of the ability of m₁ and m₄ because the distance r₂ between m₁ and m₂ is shorter than the distance r₂ between m₁ and m₄. If m₁ and m₂ form a substantially complementary double-stranded chain, a loop of single-stranded chain is formed (loop 1 in FIG. 2(B)). In this case, the possibility of forming a loop of single-stranded chain by linkage between m₁ and m₂ (loop 1 in FIG. 2 (B)) is higher or not lower than the possibility of forming a loop of single-stranded chain by linkage between m₁ and m₄ (loop 2 in FIG. 2 (C)).

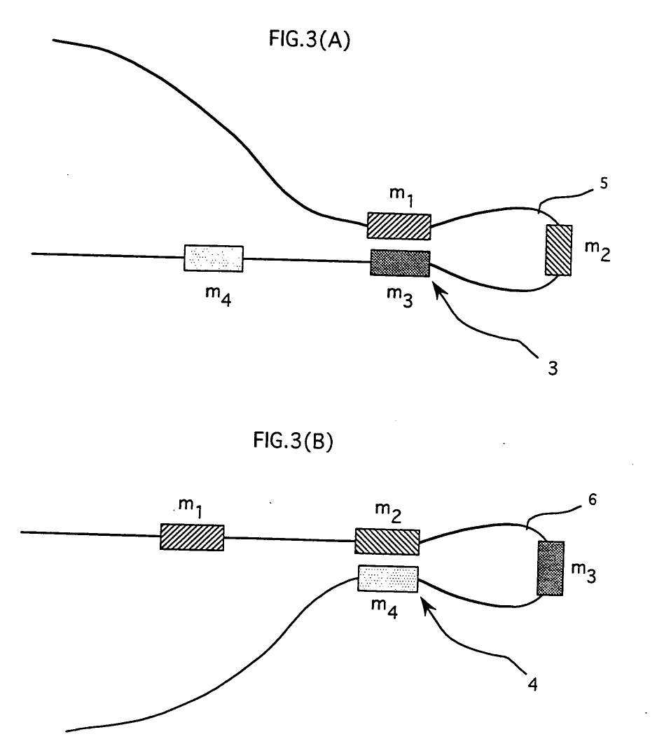

Assuming in FIG. 3 (A) and (B) that m₁ and m₂ are substantially complementary to m₃ and m₄ respectively to form double-stranded chains (stems 3 and 4) and that the length of loop 5 formed by linkage between m₁ and m₃ is equal to the length of loop 6 formed by linkage between m₂ and m₄, and if the bond energy for forming a double-stranded chain between m₁ and m₃ is higher than that between m₂ and m₄, the computer program is set such that the base pair with the higher bond energy is given a higher ability to form a substantially complementary double-stranded chain.

In FIG. 3, therefore, the possibility of forming the double-stranded chain (stem 3) between m₁ and m₃ is higher than the possibility of forming the double-stranded chain (stem 4) between m₂ and m₄.

Then, the respective values derived from the possibility of forming substantially complementary double-stranded chain is summed up for each base. In FIG. 1, the summed possibility (P1) for the base at the 1-position to be substantially complementary to a base in another region is expressed as P₁=p₁, and the summed possibility (P2) for the base at the 2-position to be substantially complementary to a base in another region is expressed as P₂=p₁+p_{2,} and P₃ for the base at the 3-position is expressed as P₃=p₂+p₃+p_{4,} and P4 for the base at the 4-position is expressed as P₄=p₃+p₄ (FIG. 1). Similarly, the summed value possibilities (P5, P6, . . . P50) are determined.

A lower value of summed possibility indicates a lower possibility of forming a substantially complementary double-stranded chain, i.e. possibility of remaining a signal-stranded chain is high, and thus a base with a lower value is preferable as a target site for antisense oligonucleotide. A region consisting of 6 continuous bases or more, preferably 10 bases or more, more preferably 15 bases or more having a low value of summed possibility is particularly hard to form a substantially complementary double-stranded chain and is thus suitable as a target site for antisense oligonucleotide. In this manner, it is possible to predict an antisense oligonucleotide target site and to design antisense oligonucleotide substantially complementary to that site.

On the basis of this design, it is possible to synthesize a natural-type oligodeoxyribonucleotide, a phosphorothioate-type oligodeoxyribonucleotide or a natural-type RNA, or an oligodeoxyribonucleotide or oligoribonucleotide modified at base, phosphate or sugar moiety.

For synthesis, the solid-phase synthesis method such as amidite method or thiophosphite method with an automatic synthesizer or the liquid-phase synthesis method such as triester method can be used. Thus obtained oligonucleotide can be purified by reverse phase or ion-exchange-type HPLC or by cartridge to prepare antisense oligonucleotide.

As for the computer program for the above method, we can use e.g. BASIC, FORTRAN, or C language or languages derived or developed therefrom.

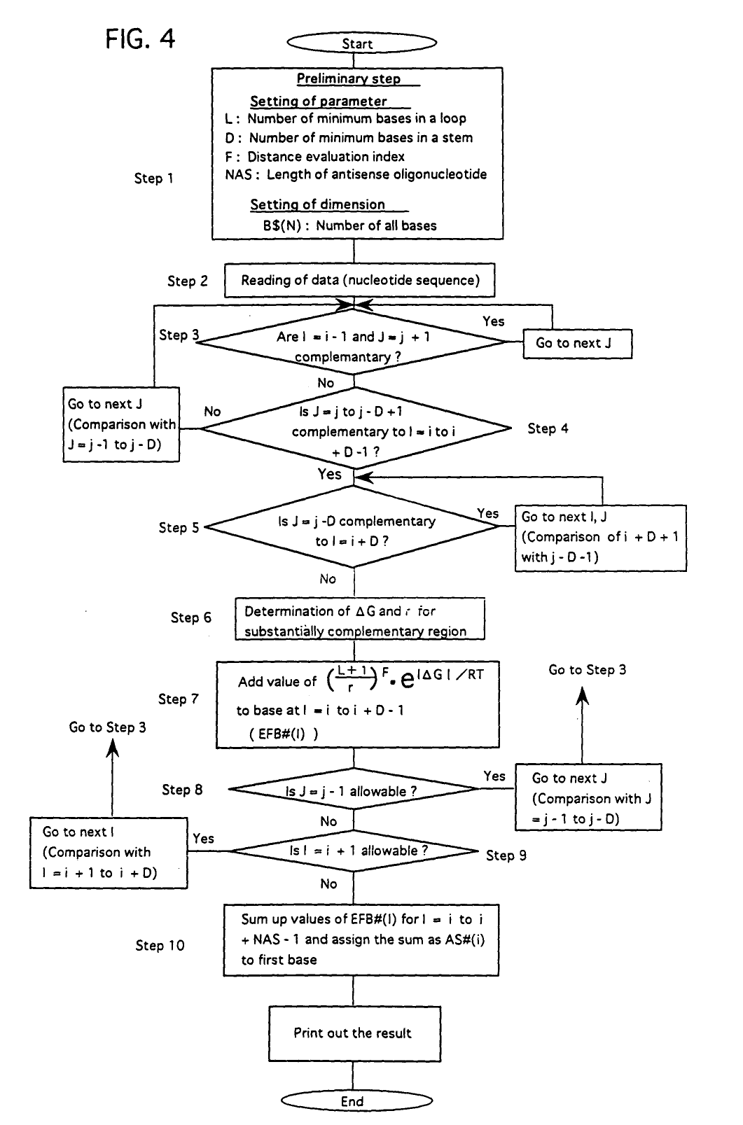

A flow chart of such a computer program is shown in FIG. 4.

In FIG. 4, parameters L, D, F and NAS as well as a dimension (number of all bases) are given predetermined values (step 1). NAS is length of antisense oligonucleotide ranging from 10 to 30, preferably 15 to 25.

Then, data (nucleotide sequence) are read (step 2).

After the data were read, complementarity is examined. The position of each base in a nucleotide sequence beginning at a specific-site base (i) is expressed as I (=i, i+1, i+2, etc.), and the position of each base in a nucleotide sequence examined for substantial complementarity to said specific site is expressed as J (=j, j−1, j−2, etc.). Before the substantial complementarity between a region beginning at I=i and a region beginning at J=j is examined, the substantial complementarity between I=i−1 and J=j+1 (where i−1<1, j+1≦N) is examined (step 3) in order to determine whether they are a part of a previously numerically expressed substantial complementary region. If a base at I=i−1 and a base at J=j+1 are substantially complementary, the substantial complementarity between a base at I=i and a base at J=j has already been examined. Therefore, it is not necessary to examine the substantial complementarity between regions beginning at I=i and J=j.

If it is judged in step 3 that it is not necessary to examine the substantial complementarity (in the case of “yes” in step 3 because I=i−1 and J=j+1 are substantially complementary), then the substantial complementary between I=i−1 and J=j is examined (this step is repeatedly carried out if the answer is “yes”). If I=i−1 and J=j+1 are not substantially complementary, it is then determined whether I=i to i+D−1 and J=j to j−D+1 are substantially complementary (step 4). If they are not substantially complementary, the step of the program returns to step 3 and the substantial complementarity between I=i−1 and J=j is examined, and if they are not substantially complementary, the substantial complementarity between I=i to i+D−1 and J=j−1 to j−D is examined in the same manner as in I=i to i+D−1 and J=j to j−D+1. If I=i to i+D−1 and J=j to j−D+1 are substantially complementary, the substantial complementarity between I=i+D and J=j−D is examined, and if they are substantially complementary, the substantial complementarity between I=i+D+1 and J=j−D−1 (step 5) is examined, and this step is repeatedly prosecuted until there appear bases which are not substantially complementary. The double-stranded chain formed by the substantially complementary regions thus obtained is examined for its ΔG and r (step 6). ΔG is determined using the nearest neighbor parameters (see Table 1), and r is expressed as the distance (in terms of number of bases) between the nearest sites in the substantially complementary chain regions. Then, the ability of the resulting complementary regions to form a substantially complementary double-stranded chain is calculated by substituting these values in the above formula I (step 7). The value thus obtained is assigned to the respective bases (bases at i to i+D−1 (or i to i+D+x) and bases at j to j−D+1 (or j to j−D−x)) in the substantially complementary region by adding this value to their previous values if any (EFB#(I) and EFB#(J)), where x=0 or a positive integer, I=i to i+D−1 or i to i+D+x, and J=j to j−D+1 or j to j−D−x.

Then, it is judged whether it is allowable to examine the substantial complementarity between a nucleotide sequence beginning at I=i and a nucleotide sequence beginning at J=j−1 (step 8). If (j−D)−(i+D−1) is not less than L+1, the answer is “yes”, otherwise “no”.

If the answer in step 8 is “no”, it is then judged whether I=i+1 is allowable (step 9). That is, if the sum of number i and number D exceeds the number of all bases, then I=i+1 is not allowable. If the answer in step 9 is “yes”, step 3 is repeatedly prosecuted to examine bases at I=(i+1) to (i+D) for their complementarity. If the answer in step 9 is “no”, the summed values (EFB#(I)) assigned to the respective bases in an NAS sequence in the position I (i.e. a region at i to (i+NAS−1)) are summed up and assigned to said NAS sequence (step 10). The value of AS#(i) is the summed ability of the NAS nucleotide sequence (a region of from i to (i+NAS−1)) to form a substantially complementary chain and this value can be used as an indication of its effectiveness as antisense oligonucleotide.

As is evident from the foregoing, a nucleotide sequence with a less value of AS#(i) can be expected to serve as effective antisense oligonucleotide.

After step 10 was finished, the result is printed out.

The effect of the present invention is as follows:

According to the present invention, a target site for antisense oligonucleotide can be predicted reliably for a target RNA nucleotide sequence without conducting any experiment, and the method of the present invention can be used to prepare oligonucleotide useful in biochemistry and molecular biology, particularly antisense oligonucleotide useful as therapeutic agents, diagnostic agents and agents for research purposes.

### EXAMPLES

The present invention is described in more detail by reference to Examples. The present invention, however, is not limited to Examples.

### Example 1

A region (635 bases between the 38- and 672-positions in SEQ ID NO:1) containing a nucleotide sequence coding for human-derived vascular endothelial growth factor (VEGF121, referred to hereinafter as “VEGF”) in a plasmid was evaluated by a BASIC program for the ability of its respective bases to form a substantially complementary double-stranded chain.

In this example, the parameters were set at L=4, D=4, F=6, and NAS=20 in the flow chart in FIG. 4.

According to these parameters, the whole nucleotide sequence of the above 635-base mRNA was examined for the complementarity between a specific region of 4 or more bases and another site apart by 5 or more bases from said specific region.

A specific region in the nucleotide sequence between the 38- and 672-positions in SEQ ID NO:1 was examined for its ability to form a substantially complementary double-stranded chain with another region. This ability was numerically expressed where a higher ability to form a substantially complementary double-stranded chain was given a larger value, and the value thus obtained was assigned to the corresponding bases. The values assigned to each base for its substantial complementarity to all other regions were summed up. In addition to the base pairs of G and C and of A and U, the base pair of G and U was also assumed to be a base pair forming a substantially double-stranded chain.

A higher ability to form a substantially double-stranded chain was also given when the base pair has higher Gibbs free energy as calculated in the nearest neighbor model (S. M. Freier et al., Proc. Natl. Acad. Sci. USA, 83, 9373-9377 (1986)). The values determined by Naoki Sugimoto et al. was used as the parameter in the nearest neighbor model (Table 1). The highest ability to form a substantially double-stranded chain was given when the distance between the nearest bases in substantially complementary regions were apart by 5 bases, and a less ability was given as the distance increases. Specific sites were set such that they were apart by 1 base from each other in the sequence, and a sequence beginning at each specific site was examined for the ability to form a substantially double-stranded chain. The sum of values assigned to each base was determined, then the summed values of 20 bases were added together and assigned to the base at the lowest-number position in said 20 bases.

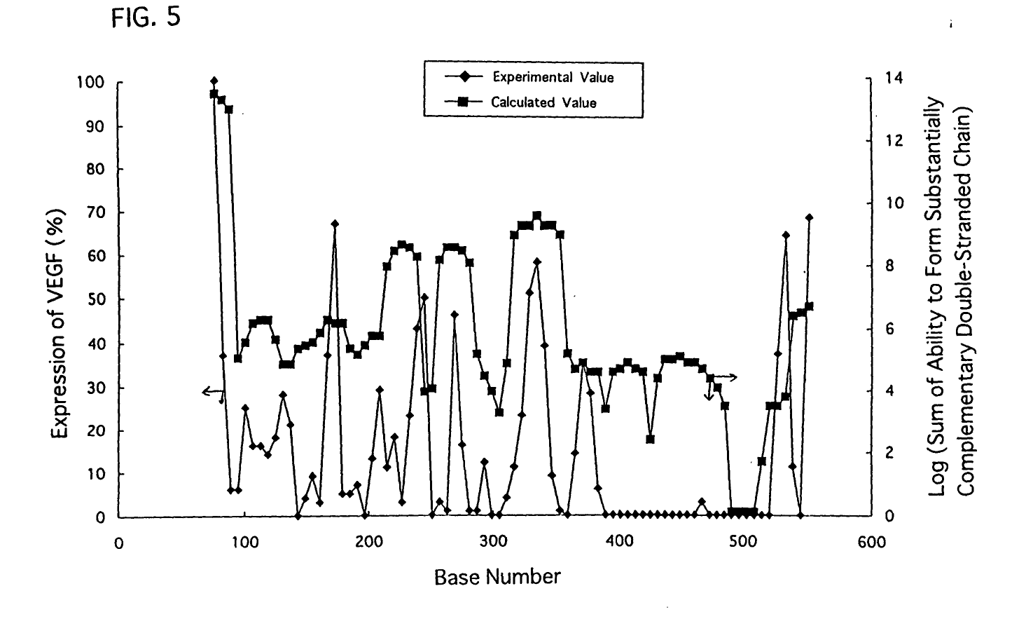

The logarithms of some values thus obtained were plotted against base number (▪ in FIG. 5). In FIG. 5, experimental results obtained using a cell-free transcription and translation system (shown in ⋄; see W096/00286) are also shown. A larger value is given on the ordinate in this graph when the ability to form a substantially double-stranded chain as determined by the calculation is higher or when the degree of expression of VEGF as determined by the experiment is higher. If there is correlation between the two results, their plots must have similar patterns.

As can be seen from FIG. 5, both the plots are roughly consistent. That is, the expression of VEGF is significantly inhibited by antisense oligonucleotide to the region at the 400- to 500-positions, while the ability of this region to form a substantially complementary double-stranded chain is low. Further, the expression of VEGF is high in the experimental results when antisense oligonucleotides beginning at bases 77, 173, 209, 245, 269, 335, 371 and 533 are used, while the ability of nearly all of them to form a substantially complementary double-stranded chain is high.

As for nucleotides with NAS (=20) bases having a logarithm of 5.5 or less (on the right ordinate) as the sum of the respective values of their bases for the ability to form a substantially complementary double-stranded chain, 82% of them brought about 10% or less expression of VEGF, and thus it is evident that both the results are in good correlation. Therefore, effective antisense oligonucleotide can be easily obtained by preparing a sequence complementary to a region with a low logarithm (5.5 or less) using a known synthetic method etc.

### Example 2

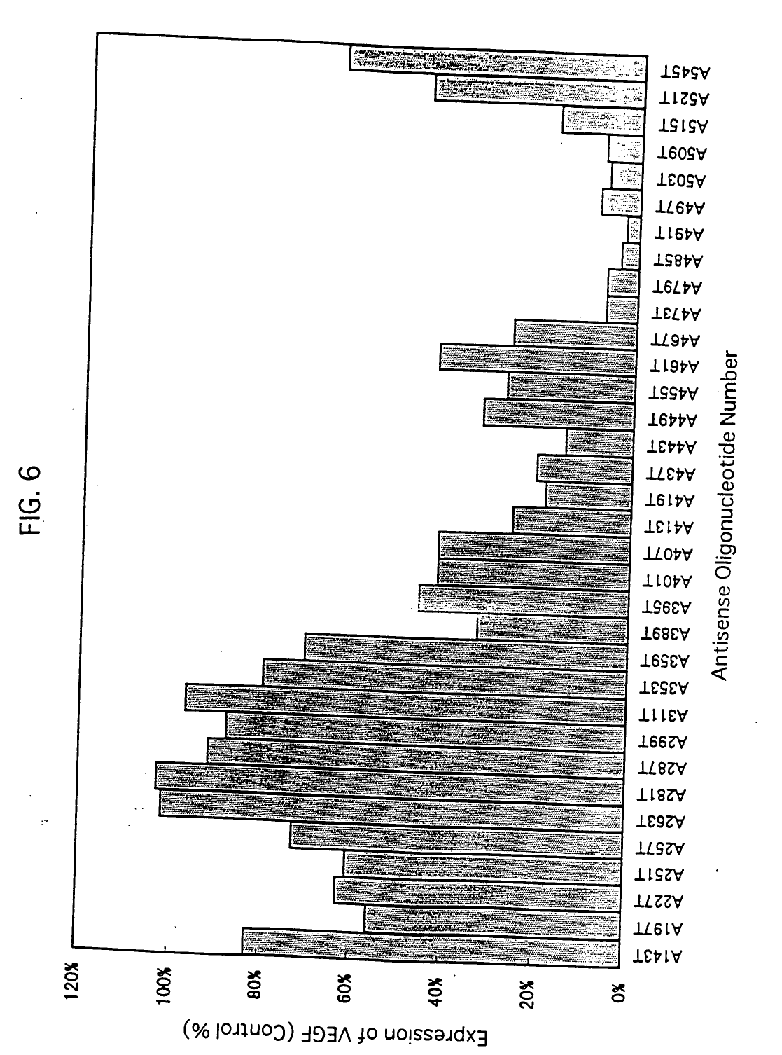

For cases where the inhibition of expression of VEGF in the cell-free transcription and translation system shown in Example 1 was significant (⋄ in FIG. 5), the production of VEGF was examined similarly except the concentrations of the antisense oligonucleotide and RNase H were ⅕ (80 nM) and 1/50 (0.0092 unit/μl) of concentrations in Example 1, respectively (FIG. 6). A143T herein used is antisense oligonucleotide (natural-type oligo-DNA) against a 20-mer beginning at the 143- position in SEQ ID NO:1. Similarly, A197T, A227T etc. are antisense oligonucleotide (natural-type oligo-DNA) against 20-mers beginning at the 197-position and the 227-position etc. in SEQ ID NO:1. The results indicated that as shown in FIG. 6, the expression of VEGF was inhibited in the presence of A473T, A479T, A485T, A491T, A497T, A503T, and A509T, among which A485T and A491T exhibited particularly strong inhibition, indicating that their antisense effect was significant. Thus results agreed well with the calculated results in FIG. 5 (the lowest value (▪) is indicated with A491T to A509T), and the method herein proposed can be used to identify a site having antisense effect. The antisense oligonucleotide shown in FIG. 6 did not inactivate the cell-free transcription and translation system itself, because in a similar experiment using a plasmid having a nucleotide sequence coding for luciferase in place of VEGF, the expression of luciferase was not inhibited. Therefore, it could be concluded that the inhibitory effect on expression as shown in FIG. 6 can be contributed to antisense effect.

### Example 3

The ability of a nucleotide sequence (including its 5′-upstream and 3′-downstream regions) coding for human-derived VEGF to form a substantially complementary double-stranded chain was evaluated in the manner as described in Example 1. The nucleotide sequence herein used is a sequence consisting of 1873 bases prepared by reference to a literature (J. Biol., 266, 11947-11954 (1991) and Science 246, 1309-1312 (1989)), and its nucleotide sequence is shown in SEQ ID NO:2. The first base in SEQ ID NO:2 (origin of transcription) was assigned as 63-position for correspondence to SEQ ID NO:1. Accordingly, the origin of translation was 1101-position corresponding to the 101-position in SEQ ID NO:1. The results indicated that the 7 nucleotide sequences shown in Table 2 are prospective antisense oligonucleotide. They were synthesized by an automatic synthesizer using the phosphoroamidite method and examined for their antisense effect on human lung cancer-derived A549 cells and human fibroblast-derived HT1080.

TABLE 2

| | | | |
|---|---|---|---|
| | | | Inhibitory |
| Antisense | | | Effect on |
| Oligo− | Nucleotide Sequence | SEQ | VEGF |
| nucleo- | of Antisense | ID | Expression |

| | | | | |
|---|---|---|---|---|
| tide # | Oligonucleotide | NO: | A549 | HT1080 |

| | | | | |
|---|---|---|---|---|
| U0370T-S | ACCTCTTTCCTCTTTCTGCT | 4 | + | ++ |
| U0406T-S | CTCTCTCTTCCTCGACTTCT | 5 | ++ | ++ |
| U0413T-S | ACCCCGTCTCTCTCTTCCTC | 6 | ++ | |
| U0853T-S | CTCCTCTTCCTTCTCTTCTT | 7 | ++ | ++ |
| U1598T-S | GTTCTGTATCAGTCTTTCCTG | 8 | + | + |
| U1676X-S | CTTCATTTCAGGTTTCTGGATTAA | 9 | + | |
| A485T-S | TCTTTCTTTGGTCTGCATTC | 10 | + | + |
| (1485T-S) | | | | |

A549 cells were cultured in MEM medium containing 10% FBS in a 48-well plate in a 5% CO₂ atmosphere at 37° C. When the number of cells reached about 1 to 3×10⁵ cells/well, they were incubated for 2 hours in a 5% CO₂ atmosphere at 37° C. in OPTI-MEM medium (in the absence of serum) containing 5.25 μg/200 μl (about 15 μM) Tfx-50 (Promega) and 2.3 μM antisense oligonucleotide. Thereafter, the medium was exchanged with fresh DMEM medium containing 10% FBS, and they were incubated for 3 hours in a 5% CO₂ atmosphere at 37° C. during which VEGF was released into the medium. The amount of VEGF was determined by the ELISA method using an anti-VEGF polyclonal antibody as primary antibody and a peroxidase-labeled anti-VEGF polyclonal antibody as secondary antibody (W096/00286).

HT1080 cells were cultured in MEM medium containing 10% FBS in a 48-well plate in a 5% CO₂ atmosphere at 37° C. When the number of cells reached about 4 to 7×10⁴ cells/well, they were incubated for 2 hours in a 5% CO₂ atmosphere at 37° C. in OPTI-MEM medium (in the absence of serum) containing 2.5 to 3.5 μg/200 μl (about 7 to 10 μM) Tfx-50 (Promega) and 1.6 to 2.3 μM antisense oligonucleotide. Thereafter, the medium was exchanged with fresh DMEM medium containing 10% FBS, and the cells were incubated for 2 hours in a 5% CO₂ atmosphere at 37° C., during which VEGF was released into the medium. The amount of VEGF was determined by the ELISA method using an anti-VEGF polyclonal antibody as primary antibody and a peroxidase-labeled anti-VEGF polyclonal antibody as secondary antibody (W096/00286).

Table 2 shows the results where “+” is given when the production of VEGF was significantly inhibited and “++” is given when the amount of VEGF produced was 50% or less as compared with the production of VEGF in the presence of the phosphorothioate-type oligo-DNA (RA419T-S having the nucleotide sequence CTAGACTGTGTGTTCTGGAG (SEQ ID NO:3)) as the control. As is evident from the results, every antisense oligonucleotide selected by calculation significantly inhibited the production of VEGF, and about half of the examined 7 sequences inhibited 50% or more expression of VEGF as compared with the control. Because a decrease in the number of both the cells was only 30% or less as compared with the case where the phosphorothioate-type oligo-DNA was not added, it was confirmed that the antisense effect was not caused by toxicity.

### Example 4

The ability of a luciferase gene to form a substantially complementary double-stranded chain was evaluated in the manner as described in Example 1. As the gene coding for luciferase, a nucleotide sequence consisting of 1150 bases shown in SEQ ID NO:11 was calculated. Here, the first base in SEQ ID NO:11 was assigned as 38-position, and the last base as 1187-position. 5 sequences (A653T, A960T, A996T, A1405T, and A1446T) which were identified by calculation as prospective candidate for antisense oligonucleotide and 50 randomly selected sequences (A085T to A575T) which were apart by 10 bases from one another were examined for their ability to inhibit the expression of luciferase in the same cell-free transcription and translation system as in Example 1. The results are shown in Table 3.

TABLE 3

| | | Position of | Expression |
|---|---|---|---|
| NO. | Designation | Nucleotide | Ratio |
| 1 | A085T | 85-104 | 4% |
| 2 | A095T | 95-114 | 9% |
| 3 | A105T | 105-124 | 11% |
| 4 | A115T | 115-134 | 23% |
| 5 | A125T | 125-144 | 23% |
| 6 | A135T | 135-154 | 6% |
| 7 | A145T | 145-164 | 11% |
| 8 | A155T | 155-174 | 12% |
| 9 | A165T | 165-184 | 3% |
| 10 | A175T | 175-194 | 5% |
| 11 | A185T | 185-204 | 14% |
| 12 | A195T | 195-214 | 8% |
| 13 | A205T | 205-224 | 13% |
| 14 | A215T | 215-234 | 7% |
| 15 | A225T | 225-244 | 10% |
| 16 | A235T | 235-254 | 10% |
| 17 | A245T | 245-264 | 10% |
| 18 | A255T | 255-274 | 112% |
| 19 | A265T | 265-284 | 101% |
| 20 | A275T | 275-294 | 21% |
| 21 | A285T | 285-304 | 8% |
| 22 | A295T | 295-314 | 14% |
| 23 | A305T | 305-324 | 60% |
| 24 | A315T | 315-334 | 10% |
| 25 | A325T | 325-344 | 4% |
| 26 | A335T | 335-354 | 7% |
| 27 | A345T | 345-364 | 33% |
| 28 | A355T | 355-374 | 17% |
| 29 | A365T | 365-384 | 23% |
| 30 | A375T | 375-394 | 13% |
| 31 | A385T | 385-404 | 12% |
| 32 | A395T | 395-414 | 35% |
| 33 | A405T | 405-424 | 1% |
| 34 | A415T | 415-434 | 12% |
| 35 | A425T | 425-444 | 25% |
| 36 | A435T | 435-454 | 12% |
| 37 | A445T | 445-464 | 20% |
| 38 | A455T | 455-474 | 26% |
| 39 | A465T | 465-484 | 8% |
| 40 | A475T | 475-494 | 11% |
| 41 | A485T | 485-504 | 6% |
| 42 | A495T | 495-514 | 3% |
| 43 | A505T | 505-524 | 6% |
| 44 | A515T | 515-534 | 24% |
| 45 | A525T | 525-544 | 12% |
| 46 | A535T | 535-554 | 5% |
| 47 | A545T | 545-564 | 2% |
| 48 | A555T | 555-574 | 5% |
| 49 | A565T | 565-584 | 12% |
| 50 | A575T | 575-594 | 22% |
| 51 | A653T | 653-672 | 1% |
| 52 | A960T | 960-979 | 6% |
| 53 | A996T | 996-1015 | 5% |
| 54 | A1405T | 1405-1424 | 2% |
| 55 | A1446T | 1446-1465 | 33% |

In Table 3, antisense oligonucleotide numbers, such as A653T etc., have the same meaning as in the previous patent (W096/00286) or as described above, and A653T refers to antisense oligonucleotide (natural-type oligo-DNA) against a 20-mer beginning at the 653-position in SEQ ID NO:11. The first base in SEQ ID NO:11 was assigned as 38-position, and the last base as 1187-positions. That is, the nucleotide sequence of A653T is CATTATCAGTGCAATTGTTT (SEQ ID NO:12). Similarly, A960T and A990T refer to antisense oligonucleotide (natural type oligo-DNA) against 20-mers beginning respectively at the 960-and 990-positions in SEQ ID NO:11.

As shown in Table 3, the 5 nucleotide sequences predicted to be suitable antisense oligonucleotide by calculation results demonstrated significant inhibition as compared with the 50 randomly selected nucleotide sequences. For example, 10% or less expression was achieved by 22 (44%) of the 50 nucleotide sequences randomly selected, whereas the same degree expression was achieved by 4 (80%) of the 5 nucleotide sequences predicted to be suitable as antisense oligonucleotide.

1-7. (canceled)

8. A method for designing an antisense oligonucleotide sequence for a target mRNA or its precursor comprising the steps of:
(a) selecting all pairs of sequences on the target mRNA or its precursor complementary to each other and separated by at least three nucleotides, without independently selecting pairs which are shorter than the selected sequences;
(b) assigning a numerical value to each pair that reflects the possibility of forming a complementary double-stranded region between said pair of sequences based upon the distance between said pair of sequences and the bond energy ΔG for said pair of sequences, wherein a lower numerical value indicates a lower possibility, and wherein the numerical value increases with an increase in said bond energy and the value decreases with an increase in the distance between said paired sequences;
(c) assigning the numerical value obtained in step (b) to each nucleotide of the paired sequences;
(d) summing the numerical values, which are assigned in step (c) for all pairs of sequences selected in step (a), for each nucleotide in the target mRNA or its precursor;
(e) selecting one or more regions which consist of at least 6 contiguous nucleotides and have a low summed value relative to another region; and
(f) designing an antisense oligonucleotide complementary to said region selected in step (e).

9. The method of claim 8, wherein said bond energy for forming the complementary double-stranded region is determined by the nearest neighbor model.

10. The method of claim 8, wherein said step (a) is conducted by the steps:
(a) selecting a first sequence consisting of 2 or more nucleotides from the target MRNA or its precursor;
(b) selecting a second sequence that is complementary to the first sequence and is separated by at least three nucleotides from the first sequence;
(c) examining whether the first and second sequences can be extended to include neighboring nucleotides by checking complementarity between corresponding neighboring nucleotides of each of the first and second sequences;
(d) extending each of the first and second sequences by one nucleotide when complementarity is found in step (c);
(e) repeating steps (c) and (d) in both directions of the first and second sequences until complementarity is not found;
(f) determining the sequences thereby selected;
(g) repeating steps (b) through (f) starting at a different region from that already selected in step (b) until all complementary second sequences for said first sequence have been selected; and
(h) repeating steps (a) through (g) for all possible first sequences on the target mRNA or its precursor without selecting the same pair more than once.

11. The method of claim 8, wherein said numerical value is expressed as ((L+1)/r)^{F}·exp(|ΔG|/RT), wherein ΔG is the bond energy for forming a complementary double-stranded region, R is the gas constant, T is the absolute temperature, L is an integer from 3 to 10, r is one plus the number of nucleic acid bases between said first target region and said complementary region, with the provision that r>L+1, and F is a positive number not greater than 6.

12. The method of claim 11, wherein |ΔG| is determined by the nearest neighbor model.

13. The method of claim 11, wherein L is 4 to 6.

14. The method of claim 11, wherein L is 4.

15. The method of claim 11, wherein F is 6.

16. The method of claim 11, wherein L is 4 to 6, and |ΔG| is determined by the nearest neighbor model.

17. The method of claim 11, wherein L is 4, and |ΔG| is determined by the nearest neighbor model.

18. A method for designing an antisense oligonucleotide sequence for a target mRNA or its precursor comprising the steps of:
(a) selecting a first sequence consisting of 2 or more nucleotides in the target mRNA or its precursor;
(b) selecting a second sequence that is complementary to the first sequence that is separated by at least three nucleotides from the first sequence;
(c) examining whether the first and second sequences can be extended to include neighboring nucleotides by checking complementarity between corresponding neighboring nucleotides of each of the first and second sequences;
(d) extending each of the first and second sequences by one nucleotide when complementarity is found in step (c);
(e) repeating steps (c) and (d) in both directions of the first and second sequences until complementarity is not found;
(f) determining the sequences thereby selected;
(g) assigning a numerical value to said sequences that reflects the possibility of forming a complementary double-stranded region between said sequences based upon the distance between said sequences and the bond energy ΔG for said sequences, wherein a lower numerical value indicates a lower possibility, and wherein the numerical value increases with an increase in said bond energy and the value decreases with an increase in the distance between said paired sequences;
(h) assigning the numerical value obtained in step (g) to each nucleotide of the sequences;
(i) repeating the steps (b) through (h) starting with different region from that already selected in step (b), until all allowable second sequences for said first sequence have been selected;
(j) repeating steps (a) through (i) for all possible first sequences on the target mRNA or its precursor without selecting the same pair more than once;
(k) summing the numerical values, which are assigned in step (h) for all sequences selected in steps (a) through (j), for each nucleotide in the mRNA or its precursor;
(l) selecting one or more regions which consist of at least 6 contiguous nucleotides and have a low summed value relative to another region; and
(m) designing an antisense oligonucleotide complementary to said region selected in step (l).

19. The method of claim 18, wherein said numerical value is expressed as ((L+1)/r)^{F}exp(|ΔG|/RT), wherein ΔG is the bond energy for forming a complementary double-stranded region, R is the gas constant, T is the absolute temperature, L is an integer from 3 to 10, r is one plus the number of nucleic acid bases between said first target region and said complementary region, with the provision that r>L+1, and F is a positive number not greater than 6.

20. The method of claim 19, wherein |ΔG| is determined by the nearest neighbor model.

21. The method of claim 19, wherein L is 4 to 6.

22. The method of claim 19, wherein L is 4.

23. The method of claim 19, wherein F is 6.

24. The method of claim 19, wherein L is 4 to 6, and |ΔG| is determined by the nearest neighbor model.

25. The method of claim 19, wherein L is 4, and |ΔG| is determined by the nearest neighbor model.
